(19) United States
(12) Patent Application Publication
(10) Pub. No.: US 2025/0332357 A1
(43) Pub. Date: Oct. 30, 2025

(54) MEDICATION AUTO-INJECTORS

(71) Applicant: KILNMARK LTD, Ripley Surrey (GB)

(72) Inventor: Andrea Sweerts de Landas Wyborgh-Markus, Ripley Surrey (GB)

(21) Appl. No.: 18/855,383

(22) PCT Filed: Apr. 13, 2023
(86) PCT No.: PCT/GB2023/050999

§ 371 Date: Oct. 9, 2024

(30) Foreign Application Priority Data
Apr. 14, 2022 (GB) 2205591.7

(51) Int. Cl. A61M 5/50; A61M 5/32

(52) U.S. Cl. CPC A61M 5/5086; A61M 5/3202; A61M 2205/18; A61M 2205/581; A61M 2205/583

(57) ABSTRACT

A medication Auto-Injector comprises a main body and a cap. A compartment is located in the main body to hold medication (for example, adrenaline) and the cap is movable between a closed condition in which the medication cannot be accessed and an open condition in which the medication can be accessed to allow medication from the compartment to be injected into the body of a user. The cap incorporates electronic components that are arranged to generate an alert signal when the cap is moved out of its closed condition. The electronic components afford communication with one or more remote device 5 (for example, a smartphone). The main body and the cap have matching outer profiles.

### CROSS-REFERENCE TO RELATED APPLICATIONS

This is the U.S. National Stage application of International Application No. PCT/GB2023/050999, filed Apr. 13, 2023, which claims the benefit of priority from GB 2205591.7, filed Apr. 14, 2022. The entire contents of these priority application are incorporated by reference.

### FIELD

The present invention relates to medication Auto-Injectors.

### BACKGROUND

Patients often need to keep medication with them wherever they go, to ensure that they keep well and, in critical cases, to ensure that they keep alive. One example of this is the Adrenaline Auto-Injector (AAI), which enables a patient to inject themselves with Adrenaline in the event of an anaphylactic shock.

Anaphylaxis is a life-threatening condition and Adrenaline Auto-Injectors (AAIs), which contain lifesaving adrenaline, must be carried at all times by those whose life depends on it. One well-known brand of AAI is the EpiPen®. Other medications may also be delivered by Auto-Injectors.

It is critically important to use an AAI device properly. Most owners/users are trained once only, and they may not always be fully conscious when the AAI has to be used. Therefore, instructions on its use are critically important. People looking after young patients might not have been trained at all, or the patient might already be unconscious or unable to speak and therefore by-standers have to administer it.

### BRIEF SUMMARY

Keeping a record of the use of medications, especially such as those above, can be very beneficial, not only for a patient, but also for medical professionals.

Preferred embodiments of the present invention aim to provide medication Auto-Injectors that are generally improved in the foregoing respects.

According to one aspect of the present invention, there is provided a medication auto-injector comprising a main body and a cap, wherein a compartment is located in the main body to hold medication and the cap is movable between a closed condition in which the medication cannot be accessed and an open condition in which the medication can be accessed to allow medication from said compartment to be injected into the body of a user, wherein the cap incorporates electronic components that are arranged to generate an alert signal when the cap is moved out of its closed condition, the electronic components affording communication with one or more remote device.

Preferably, the main body has an outer profile and the cap has an outer profile that matches that of the main body.

The cap may be secured to a cover of the main body.

Preferably, said cover has an outer profile that matches those of the cap and the main body.

Preferably, the cap is arranged to be removed from the main body to allow the medication to be accessed.

The cap may be arranged to be twisted to move between its open and closed conditions.

Preferably, the electronic components are arranged to output one or more data signal that indicates one or more of the following:
a. identity of the medication
b. dispense date of the medication
c. expiry date of the medication
d. identity of the user of the medication
e. nature of allergy of the user
f. present ambient temperature
g. historical ambient temperatures
h. contact details of next of kin of the user
i. contact details of medical adviser of the user
j. contact details of pharmacy or other supplier of the medication
k. condition of a battery that powers the electronic components

Preferably, the electronic components are arranged to establish communication with one or more remote device in order to transmit said alert signal and/or one or more data signal to the one or more remote device.

Preferably, said electronic components provide GPS or other location functionality.

The invention extends to a medication Auto-Injector system comprising a medication Auto-Injector according to any of the preceding aspects of the invention, in combination with a remote device that is arranged to communicate with said electronic components for the exchange of data with said electronic components.

Preferably, the remote device stores and/or processes data relating to the medication container.

Preferably, the remote device stores and is arranged to output audible and/or visible instructions for use of the medication container.

Preferably, the remote device is configured to output audible and/or visible instructions in a local language, the location being detected by the Auto-Injector and/or the remote device.

Preferably, the remote device has a volume setting and is configured to output said instructions at a high or maximum volume, irrespective of any volume currently set by a user of the device.

Preferably, the remote device is configured to output said instructions, irrespective of whether the device has been switched to an ‘off’ condition by a user of the device.

Preferably, the remote device is configured to output said instructions, irrespective of whether the device has been set to an ‘aeroplane’ mode or the like by a user of the device.

Preferably, the remote device is configured to send a message to a supplier of the medication container in order to initiate an order for a new medication container, upon receiving said alert signal and/or a data signal indicating approaching, actual or past expiry date of the medication.

A medication Auto-Injector system as above preferably comprises a device at the supplier of the medication container to receive said message in order to initiate an order for a new medication container.

Preferably, the remote device is configured to send a message to one or more third party upon receiving said alert signal and/or a data signal indicating approaching, actual or past expiry date of the medication.

The third party may be a relative or medical adviser of the user of the medication.

Preferably, upon receipt of said alert signal, the remote device is configured to display a message that affords a user the opportunity to confirm or deny that the alert signal is in respect of a medical emergency.

Preferably, the remote device and the medication container are arranged to interact in order to provide a warning signal when the medication container is further than a predetermined distance from the remote device.

Preferably, the remote device and the medication container are arranged to interact in order to provide an indication of the location of the medication container.

The invention extends to a medication Auto-Injector or Auto-Injector system according to any of the preceding aspects of the invention, in combination with adrenaline contained within said compartment.

### BRIEF DESCRIPTION OF THE DRAWINGS

For a better understanding of the invention, and to show how embodiments of the same may be carried into effect, reference will now be made, by way of example, to the accompanying diagrammatic drawings, in which:

FIG. 1 is a diagrammatic view of an Adrenaline Auto-Injector (AAI);

FIG. 2 illustrates a smartphone;

FIGS. 3A to 3H illustrate windows that may be displayed on the smartphone; and

FIG. 4 illustrates a network of electronic devices that are arranged to communicate with one another.

### DESCRIPTION

In the figures, like references denote like or corresponding parts.

It is to be understood that the various features that are described in the following and/or illustrated in the drawings are preferred but not essential. Combinations of features described and/or illustrated are not considered to be the only possible combinations. Unless stated to the contrary, individual features may be omitted, varied or combined in different combinations, where practical.

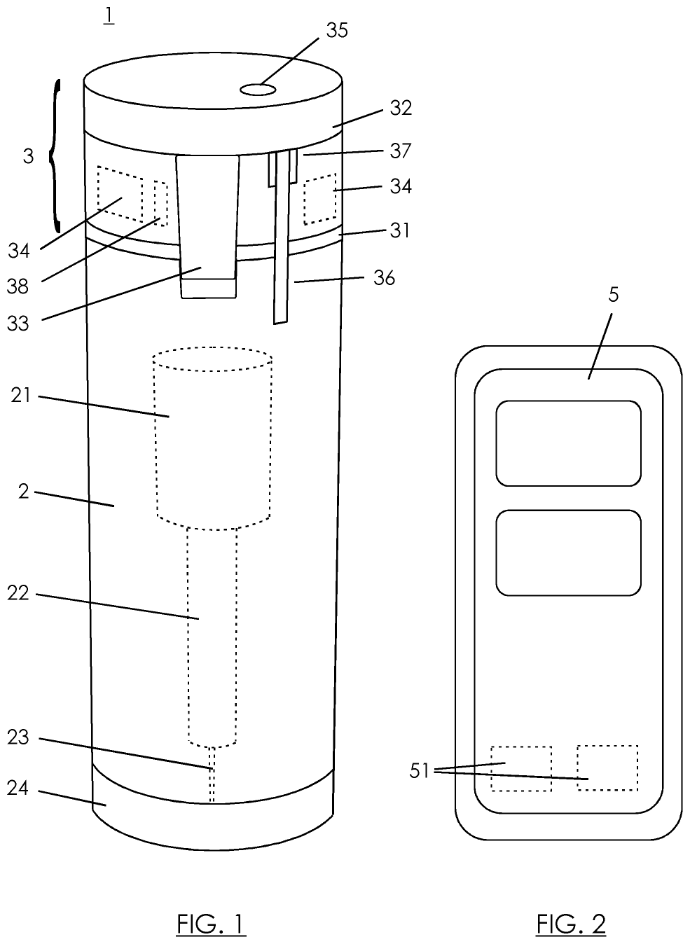

The Adrenaline Auto-Injector (AAI) 1 of FIG. 1 serves as both a medication container and medication dispenser. The medication in this case is adrenaline. The AAI 1 is generally elliptical in cross-sectional shape; it could be of other cross-sectional shapes—e.g. generally rectangular with rounded corners. The AAI 1 has a main body 2 in which there is located a compartment 21 that contains adrenaline. A delivery mechanism 22 receives adrenaline from the compartment 21 and is arranged to deliver a dose of adrenaline through an injection needle 23, via a lower part 24.

The precise configuration of the delivery mechanism 22 is not essential to an understanding of the illustrated embodiment of the invention and is therefore not described here in detail. Injection delivery mechanisms of AAIs are well known to the skilled reader.

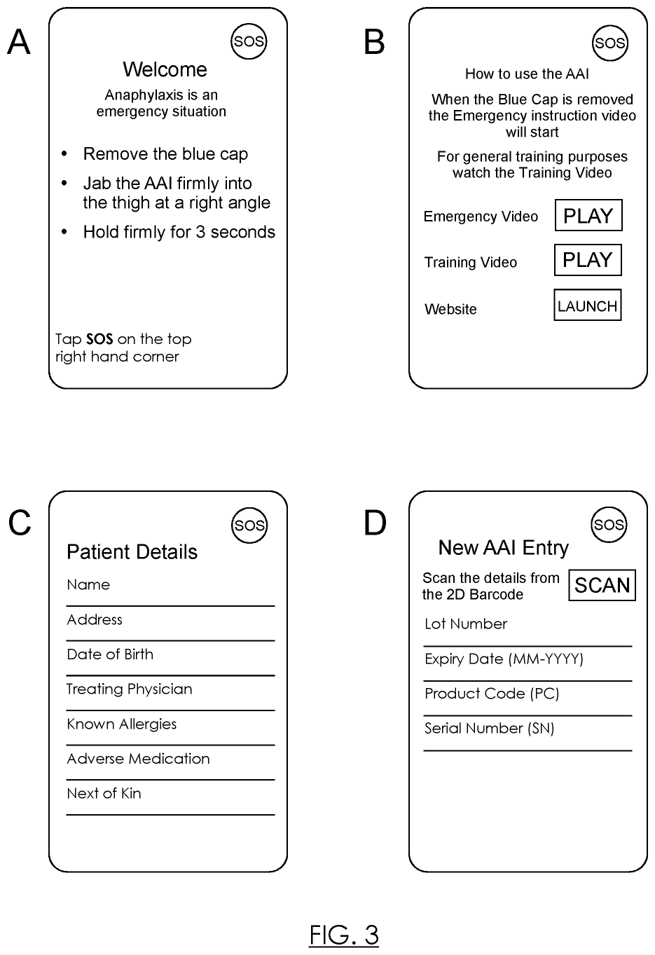

A cap 3 is secured to the top of the main body 2 and is normally in a closed condition. In this example, the cap 3 engages a cover 31 that has to be removed from the main body 2 along with the cap 3 to allow the adrenaline in the compartment 21 to be accessed by a patient, via the delivery mechanism 22. The cap 3 includes a housing 32 that is secured to the cover 31 by means of clips 33 that snap-engage a rim of the cover 31, at opposite sides of the cover 31, via cut-outs or recesses in the main body 2. Electronic components 34 are contained within the housing 32 and powered by a battery 38. A button 35 on top of the housing 32 may initiate or cancel electronic communication with a remote electronic device, such as a smartphone 5 with electronic components 51, as illustrated in FIG. 2. The electronic components 34 and 51 may include processor, memory, communications module, I/O module and so on. The electronic communication may be by way of any suitable protocol—in this example, Bluetooth®. Near Field Communication (NFC) is another option.

A pair of ribbon connectors 36 are secured at one end to the upper portion of the main body 2, one at each side of the body 2. The other ends of the ribbon connectors 36 engage respective components within the housing 32, which they access through ports 37 in the housing 32.

In order to activate the AAI 1, the cap 3 is grasped and disengaged bodily from the top of the main body 2, releasing the clips 33. Typically, the movement for disengaging the cap 3 from the AAI is a vertical lift done with the left hand, with the right hand (dominant) holding the main body 2. Or vertical lift by the right hand where the left hand is dominant. In this process, the ribbon connectors 36 remain affixed to the main body 2 and the upper ends of the ribbon connectors 36 are pulled away from the components within the housing 32 with which they were previously engaged. This disengagement is detected by the electronic components 34 within the housing 32, which generate an alert signal to indicate that the cap 3 has been moved out of its closed condition. The alert signal is transmitted to the smartphone 5 by way of the Bluetooth connection.

Once the cap 3 has been disengaged from the main body 2, the delivery mechanism 22 is unlocked and the adrenaline in the compartment 21 can be accessed and delivered. Typically, the AAI is pushed firmly against the thigh with the lower part 24 in contact with the thigh, to enable injection needle 23 to enter the thigh and deliver the adrenaline. Typically, the AAI is held in position for several seconds, to allow full delivery of the medication. Typically, these instructions are written on the AAI itself.

It is to be noted that users of AAIs will be familiar with this process of delivering adrenaline. The basic delivery function of the illustrated example is essentially unchanged. Remove the cap—push the lower part of the AAI firmly against the thigh—leave it there for several seconds—remove the AAI. All of these steps are much as usual.

However, the illustrated example provides a number of novel and advantageous features, especially when linked to the smartphone 5.

The electronic components 34 within the cap 3 include a component for measuring present temperature of the medication in the compartment 21 and/or present ambient temperature and storing the temperature in a memory of the AAI 1, afforded by the electronic components 34, along with present date and time. Also stored in a memory of the AAI 1 are upper and lower temperature limits, within which the medication must be kept. In the event of a measured present temperature being outside the upper and lower temperature limits, the electronic components 34 generate an alert.

Also stored in the memory of the AAI is the date on which the medication was dispensed, along with the expiry date of the medication. In the event of the expiry date being approached and/or overrun, the electronic components 34 generate an alert.

The memory of the AAI also stores essential data of the AAI and/or medication—such as, for example, Lot number, Product Code and Serial Number.

The electronic components 34 of the AAI monitor the condition of the battery 38 and, in the event of the battery deteriorating, generate an alert.

The electronic components 34 of the AAI include a GPS transponder.

As indicated above, the smartphone 5 communicates with the AAI to receive alerts from it. This facilitates a number of advantageous features.

First of all, upon receiving an ‘Active’ alert from the AAI to indicate that the cap 3 has been removed, the smartphone 5 displays instructions on how to use the AAI. This may be to assist the AAI owner, who may have not used the AAI before or for a long time, or to assist a bystander to administer the AAI, in the event that the AAI owner is compromised from doing so.

The instructions may be audio instructions, video instructions or both. Audio instructions are given in a very calm voice, which is important in a stressful situation. Preferably, an initial announcement is given to say “This person has an allergy to . . . (nature of allergy)”. Optionally, audio instructions are given in a local language, GPS enabling identification of the local country. Instructions in the local language may alternate with instructions in a default language that the user sets, if different. Preferably, simple and clear audio instructions are played through the smartphone 5 at high or maximum volume irrespective of any volume currently set by a user of the smartphone 5, irrespective of whether the smartphone 5 has been switched to an ‘off’ condition by a user, and/or irrespective of whether the smartphone 5 has been set to an ‘aeroplane’ mode or the like by a user.

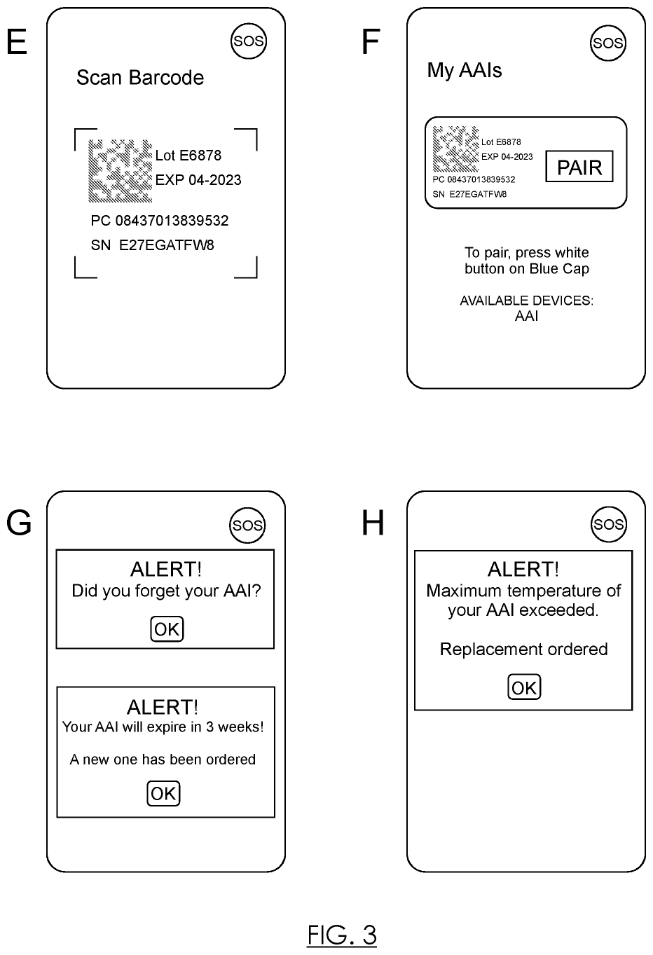

When an Active alert is not in progress, a user may access a program that is installed in the smartphone 5, to cooperate with the AAI 1. FIG. 3A illustrates a ‘Welcome’ window that may be displayed upon accessing the program. As may be seen, instructions for using the AAI are displayed simply and clearly. An option is displayed for the phone user to tap an SOS icon, which initiates playing of emergency audio/video instructions on how to use the AAI, and preferably establishes an immediate call to emergency services. The SOS icon is included on all windows that are displayed on the smartphone 5.

The smartphone 5 will typically have a number of navigation buttons to navigate through various windows and options. However, in the interests of clarity, such buttons are not included in the figures.

In the window shown in FIG. 3B, options are given to play an Emergency (Instruction) Video or, alternatively, a Training Video. The latter may be appropriate when there is no emergency situation and the AAI owner, or anyone else, wishes to refresh their understanding of how the AAI is used. Another option in the window of FIG. 3B is to launch the website of the manufacturer of the AAI, or other website such as that of an AAI supplier or adviser, where detailed information about the AAI may be found, including training information and/or an interactive training program.

In the window shown in FIG. 3C, a user may enter both personal and medical details. These details may be entered manually and/or at least partially by an automated process whereby the smartphone 5 exchanges data with a data server managed by an AAI supplier—e.g. a medical services provider and/or a pharmacy.

In the window shown in FIG. 3D, a user, medical services provider and/or a pharmacist may enter essential details of a current AAI. Instead of entering the details manually, a SCAN button gives the option to scan a barcode on an AAI or its packaging, whereby the essential details may be captured. An example of such a scan is shown in the window of FIG. 3E.

The window shown in FIG. 3F provides Bluetooth (or other communication) pairing between the smartphone 5 and the AAI 1. A sub-window in FIG. 3F identifies the AAI corresponding to that of FIG. 3E, with a PAIR button alongside. In order to pair with the AAI, both the button 35 on the cap 3 and the PAIR button of FIG. 3F are pressed. Bluetooth (and other) pairing is a widely known process.

Whilst just one sub-window with AAI details is shown in FIG. 3F, further such sub-windows may also be displayed, whereby the smartphone 5 can pair with different AAIs or other medication devices or containers.

In addition to providing potentially life-saving instructions as to use of an AAI (or other device), the smartphone 5, interacting with the AAI 1, can provide various other alerts to ensure the safety of the AAI user. Visual alerts will generally be accompanied by an audible alert.

The window shown in FIG. 3G shows a first sub-window that displays an alert to ask a user if they have forgotten their AAI. This alert may display when the smartphone 5 is at a distance from the AAI 1 that exceeds a threshold. The distance can be established via various communication techniques, including GPS location. On the assumption that the user is likely to keep their smartphone 5 close by them, an alert such as that shown is likely to indicate that the AAI has inadvertently been left somewhere. A user can tap an OK button, to acknowledge receipt of the alert.

Instead of measuring distance between AAI 1 and smartphone 5, other techniques such as geo-fencing can be used, to ensure that the AAI is not moved outside a predetermined area.

A second sub-window in FIG. 3G displays a different alert, to inform the user that their AAI will expire within a predetermined time—in this case, 3 weeks. Further such alerts can be displayed—for example, to inform the user that their AAI has expired.

A particularly useful feature of the illustrated system is that, upon generating an alert to indicate upcoming expiry of the AAI (or other medication), a message is transmitted to a pharmacy (or other medical service), upon the receipt of which a new AAI is automatically ordered for the user. The second sub-window in FIG. 3G informs the user that ‘A new one has been ordered’.

Various other alerts may be displayed on the smartphone 5. In FIG. 3H, an alert informs the user that a maximum temperature for the AAI has been exceeded. This is likely to compromise the integrity of the medication. In this example, the alert is also sent to a pharmacy to order an immediate replacement AAI.

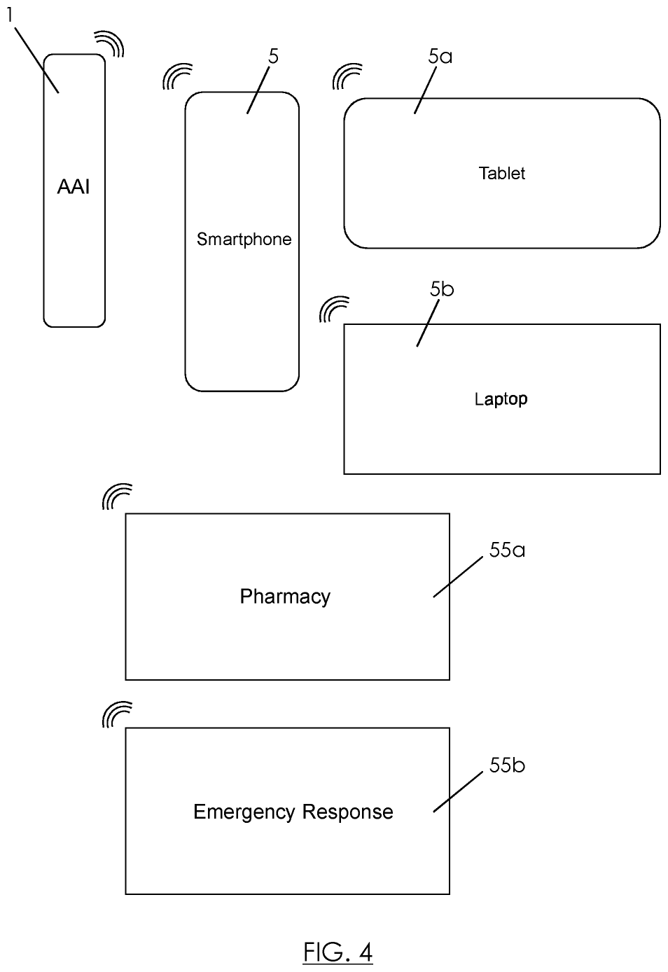

FIG. 4 illustrates a network of electronic devices arranged to communicate with one another. As indicated above, the AAI 1 and smartphone 5 interact with one another to provide various advantageous features. Typically, the communication is wireless communication. The smartphone 5 is also arranged to communicate wirelessly with various other electronic devices such as, in this example, a tablet 5a, a laptop 5b, a Pharmacy device 55a and an Emergency Response device 55B. This enables alerts to be received not only on the smartphone 5, typically owned by a user of the AAI 1, but also various other parties that may need to be informed and/or made ready to provide medical assistance. For example, next of kin may be informed by an alert when a user leaves an AAI or other device behind—such as illustrated in the first sub-window of FIG. 3G. This can be particularly important for children, including teenagers. Both next of kin and Emergency Services may be informed whenever the cap 3 is removed.

The AAI 1 may communicate with the various devices of FIG. 4, either via the smartphone 5 or directly.

A particularly useful feature of the illustrated system is that data relating to use and storage of devices such as the AAI 1 can be monitored and transmitted to a central database, preferably on an anonymous basis, where it is analysed to provide useful information that can be used to develop new devices and systems that are best suited to their purpose.

Various options may be adopted.

The ribbon connectors 36 may be replaced by one or more microswitch that is activated when the cap 3 is removed from the main body 2.

The example of FIG. 1 illustrates a ‘retrofit’ option, where the cap 3 is fitted to the cover 31 of a pre-existing AAI. The cap 3, cover 31 and main body 2 have matching outer profiles. The radial dimensions of the cap 3, cover 31 and main body 2 are substantially the same, to provide an overall outer surface that is substantially flush, apart from clips 33. There may be minor variations in radial dimensions. For example, the cap 3 may have a slightly greater radial dimension than the cover 31, which in turn may have a slightly greater radial dimension than the main body 2. For example, the cap 3, cover 31 and main body 2 may have radial dimensions within 5% or 10% of one another. Where the cap 3 is provided as original equipment, rather than a retrofit, the cover 31 may be dispensed with—that is, incorporated into the cap 3. Clips 33 on the cap 3 may then engage recessed indents in the main body 2 to provide a substantially flush outer surface and provide more secure engagement between the cap 3 and main body 2.

It will be appreciated that, in either of the above cases, the cap 3 is relatively small compared to the overall size of the AAI 1. This means that a user can readily carry around an AAI that is not significantly greater in size, which is a great practical advantage.

By way of example, as seen in FIG. 1, a prototype cap 3 has a width from left to right of the order of 30 mm, a depth from front to back of the order of 25 mm and height from top to bottom, including clips 33, of the order of 20 mm. Miniaturisation in production can reduce these dimensions, particularly the height, considerably.

The smartphone 5 (or other device) may provide a ‘Find my AAI’ option. Technology for such functions is known—for example, Apple Airtag® and similar Android® products that can locate items for an owner, interact with smartphones, and indicate if an item has been left behind. A suitable transponder and/or circuitry is preferably included in the electronic components 34, to provide such functionality.

If the cap 3 gets dislodged sufficiently to be detected by the electronic components 34, but not sufficiently to indicate that it has been fully removed, an alert signal may be generated to indicate that the AAI 1 has been damaged. A replacement AAI has to be ordered and this is preferably ordered automatically—e.g. as outlined above. In the event of a damage alert, a user is given an option to indicate that ‘This is not an emergency’.

Upon the cap 3 being removed and the Emergency Video playing, a message may be displayed on the smartphone 5 to allow the user to indicate that the cap 3 has been removed accidentally and that there is therefore no emergency situation. This prevents unnecessary alarm to next of kin and/or Emergency Services. A replacement AAI may be ordered automatically—e.g. as outlined above.

The AAI 1 and smartphone 5 may cooperate to indicate when battery 38 is running low. This may be by way of an alert and/or a continuous indication on the smartphone 5 of battery condition. It is anticipated that the life of battery 38 may match the life of the medication sufficiently well that the battery 38 does not need replacement before the expiry date of the medication. However, if the battery 38 runs down sooner than expected, an alert may be generated whereupon the battery may be replaced (if possible) and/or a replacement AAI may be ordered automatically—e.g. as outlined above.

In this specification, the verb “comprise” has its normal dictionary meaning, to denote non-exclusive inclusion. That is, use of the word “comprise” (or any of its derivatives) to include one feature or more, does not exclude the possibility of also including further features. The word “preferable” (or any of its derivatives) indicates one feature or more that is preferred but not essential.

AI or any of the features disclosed in this specification (including any accompanying claims, abstract and drawings), and/or all or any of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1.-50. (canceled)

51. A cap for a medication Auto-Injector, the auto-injector comprising a main body having a compartment to hold medication, wherein:
the cap is movable between a closed condition and an open condition on the medication auto-injector, in which:
in the closed position the medication cannot be accessed, and
in the open condition the medication can be accessed to allow medication from the compartment to be injected into the body of a user, and
wherein the cap incorporates electronic components that are arranged to generate an alert signal when the cap is moved out of its closed condition, the electronic components affording communication with one or more remote devices.

52. The cap for a medication Auto-Injector according to claim 51, wherein the main body of the medication auto-injector has an outer profile and the cap has an outer profile that matches that of the main body.

53. The cap for a medication Auto-Injector according to claim 51, wherein the cap is secured to a cover of the main body, and wherein said cover has an outer profile that matches those of the cap and the main body.

54. The cap for a medication Auto-Injector according to claim 51, wherein the cap is arranged to be twisted to move between its open and closed conditions.

55. The cap for a medication Auto-Injector according to claim 51, wherein the electronic components are arranged to output one or more data signals that indicate one or more of:
a. an identity of the medication;
b. a dispense date of the medication;
c. an expiry date of the medication;
d. an identity of the user of the medication;
e. a nature of an allergy of the user;
f. a present ambient temperature;
g. a historical ambient temperature;
h. a contact detail of next of kin of the user;
i. a contact detail of a medical adviser of the user;
j. a contact detail of a pharmacy or other supplier of the medication; or
k. a condition of a battery that powers the electronic components.

56. The cap for a medication Auto-Injector according to claim 51, wherein the electronic components are arranged to establish communication with the one or more remote devices in order to transmit said alert signal and/or one or more data signal to the one or more remote devices.

57. The cap for a medication Auto-Injector according to claim 51, wherein said electronic components provide a GPS or other location functionality.

58. The cap for a medication Auto-Injector according to claim 51, wherein the medication auto-injector is in combination with a first remote device of the one or more remote devices that is arranged to communicate with said electronic components for the exchange of data with said electronic components,
wherein the first remote device stores and/or processes data relating to the medication container, and
wherein the first remote device stores and is arranged to output audible and/or visible instructions for use of the medication container.

59. The cap for a medication Auto-Injector according to claim 58, wherein the first remote device is configured to output audible and/or visible instructions in a local language, the location being detected by the Auto-Injector and/or the first remote device,
wherein the first remote device has a volume setting and is configured to output said instructions at a high or maximum volume, irrespective of any volume currently set by a user of the device,
wherein the first remote device is configured to output said instructions, irrespective of whether the device has been switched to an ‘off’ condition by a user of the device, and
wherein the first remote device is configured to output said instructions, irrespective of whether the device has been set to an ‘aeroplane’ mode or the like by a user of the device.

60. The cap for a medication Auto-injector according to claim 59, wherein the first remote device is configured to send a message to a supplier of the medication container in order to initiate an order for a new medication container, upon receiving said alert signal and/or a data signal indicating approaching, actual or past expiry date of the medication, and
wherein a device at the supplier of the medication container is configured to receive said message in order to initiate an order for a new medication container.

61. The cap for a medication Auto-Injector according to claim 58, wherein the first remote device is configured to send a message to a third party upon receiving said alert signal and/or a data signal indicating approaching, actual or past expiry date of the medication, and
wherein the third party is a relative or medical adviser of the user of the medication.

62. The cap for a medication Auto-Injector according to claim 58 wherein, upon receipt of said alert signal, the first remote device is configured to display a message that affords a user the opportunity to confirm or deny that the alert signal is in respect of a medical emergency.

63. The cap for a medication Auto-Injector according to claim 58, wherein the first remote device and the medication container are arranged to interact in order to provide a warning signal when the medication container is further than a predetermined distance from the first remote device.

64. The cap for a medication Auto-Injector according to claim 58, wherein the first remote device and the medication container are arranged to interact in order to provide an indication of the location of the medication container.

65. The cap for a medication Auto-Injector according to claim 51, wherein the compartment is configured to contain adrenaline.
